(19) United States
(12) Patent Application Publication
(10) Pub. No.: US 2007/0031839 A1
(43) Pub. Date: Feb. 8, 2007

(54) Identification of pharmaceutical targets

(76) Inventors: Bernd Schuermann, Haimhausen (DE); Martin Stetter, Munich (DE)

Correspondence Address: STAAS & HALSEY LLP, SUITE 700, 1201 NEW YORK AVENUE, N.W., WASHINGTON, DC, 20005, US

(21) Appl. No.: 10/571,669

(22) PCT Filed: Aug. 18, 2004
(86) PCT No.: PCT/EP04/51835

§ 371 Date: Mar. 13, 2006

(30) Foreign Application Priority Data
Sep. 12, 2003 (DE) 103 42 274.9

(51) Int. Cl. C12Q 1/68; G06F 19/00

(52) U.S. Cl. 435/6; 702/20

(57) ABSTRACT

An equivalence relationship is created between a) the functional network of the genome and proteome and b) a neuronal network. Both networks represent highly cross-linked feedback systems. The equivalence relationship makes it possible to model the functional network of proteins of and genes by an equivalent artificial neuronal network. The dynamic interaction of genes and regulatory proteins is modeled by a dynamic neuronal network. The method uses information obtained in a temporal sequence of gene expression patterns for identification of causal regulatory correlations, thereby enabling target proteins to be identified on a systematic basis.

### CROSS REFERENCE TO RELATED APPLICATIONS

This application is based on and hereby claims priority to PCT Application No. PCT/EP2004/051835 filed on Aug. 18, 2004 and German Application 10342274.9 filed Sep. 12, 2003, the contents of which are hereby incorporated by reference.

### BACKGROUND OF THE INVENTION

It is estimated that the human genome comprises 20,000 to 80,000 genes, which contain the genetic code for around one million proteins. In the specialized body cells only subsets of all genes are actually read off (expressed) in each case. The totality of the proteins created in this way is referred to as the proteome of this cell. The interplay of the proteins with each other as well as with the DNA represents the most important part of the machinery which underlies the development of the human body from the fertilized egg cell as well as all body functions. From the information technology standpoint the genome thereby represents a procedural code for the structure and function of the human body.

Many illnesses and malfunctions of the body are attributable to faults in the functional network of genome and proteome. Thus a plurality of medicaments operate as agonists or antagonists of specific target proteins, i.e. they strengthen or weaken the function of a protein with the aim of returning the function of the regulatory network formed from proteome and genome back into a normal functional mode. These targets have previously been derived using heuristic principles from biochemical considerations. It is often unclear in such cases whether the malfunction of a protein represents the actual cause of the illness or only one of the symptoms of a hidden mis-regulation at another point of the network.

The simulation of nerve cells (neurons) and their biological functionality by artificial neurons is known from Zell, A., “Simulation Neuronaler networks” (Simulation of Neuronal Networks), P. 35 to 51, P. 55 to 86, Addison-Wesley Longman Verlag GmbH, 1994, 3rd unamended reprint, R. Oldenbourg Verlag, ISBN 3-486-24350-0, 2000 (“Zell pages 35 to 51 and 55-86”) and Patent Application PCT/DE02/03381 (“PCT03381”).

Making a distinction between two types of synapses or nerve cells, an excitatory synapse 251 or nerve cell and an inhibiting synapse 252 or nerve cell is further known from Zell pages 35 to 51 and 55-86.

Inhibitory synapses 252 reduce electrical potentials to be transmitted or forwarded, excitatory synapses 251 increase electrical potentials to be transmitted.

Further information about the structure and functionality of a nerve cell as well as for nerve conductance is given in Zell pages 35 to 51 and 55-86.

Furthermore an artificial nerve cell (artificial neuron) which emulates a (biological) nerve cell is known from Zell pages 35 to 51 and 55-86 and PCT03381.

At first glance such an artificial neuron is a mathematical mapping, which, in accordance with the transmission behavior of the biological nerve cell, maps an input variable of the artificial neuron onto an output variable of the artificial neuron.

In compliance with the biological template an artificial neuron has three components: the cell body, the dendrites, which sum input signals into the artificial neuron, the axon, which forwards the output signal of the artificial neuron to the outside, branches and comes into contact with the dendrites of subsequent artificial neurons via synapses.

A strength of a synapse or the type of the synapse is mostly represented by a numeric value or by its leading sign. This value is referred to as the connection weight.

In accordance with the biological template a transmission behavior or the mapping behavior of an artificial neuron can be mapped as described in Zell pages 35 to 51 and 55-86 and PCT03381.

Further information about artificial neurons and their functionality is given in Zell pages 35 to 51 and 55-86 and PCT03381.

The linkage of individual neurons with each other is further known from Zell, A., “Simulation Neuronaler networks” (Simulation of Neuronal Networks), P. 87 to 114, Addison-Wesley Longman Verlag GmbH, 1994, 3rd unamended reprint, R. Oldenbourg Verlag, ISBN 3-48624350-0, 2000 (“Zell pages 87-114”) and PCT03381. Such an arrangement with linked neurons is referred to as a neuronal network. The basics of neuronal networks, for example different types of neuronal networks, training methods for neuronal networks, references to biological nerve cell arrangements, are described in Zell pages 87-114.

An application of a mean-field model to the description of a complex system is known from J. J. Binney, N. J. Dowrick, A. J. Fisher, M. E. J. Newman, “The Theory of Critical Phenomena”, Chap. 6: Mean-Field Theory, Clarendon Press Oxford 1992 and PCT03381. With a mean-field model stochastic interaction influences between components of a system are approximated by a mean interaction influence. This allows stochastic systems which cannot be described analytically to be reduced to describable, deterministic systems.

The application of the mean-field model to the description of a neuron structure is known from C. Koch, I. Segev (Hrsg), “Methods of Neural Modeling: From Ions To Networks”, Chap 13: D. Hansel and H. Sompolinsky: “Modeling Feature Selectivity in Local Circuits”, MIT Press, Cambridge, 1998 and PCT03381.

### SUMMARY OF THE INVENTION

One possible object of the invention is to improve the identification of proteins which are suitable as targets for treatment without medicaments of genetic diseases or problems.

The inventors propose to determine a plurality of gene expression patterns of similar types of cells or of a tissue, with an expression rate of the genes of the cell being determined in each case. The plurality of gene expression patterns is determined such that the chronological sequence of the gene expression pattern of the cell can be at least partly reconstructed. A dynamic model of the regulatory network made up of genome and proteome of the cell is formed by forming an equivalent neuronal network in the following way:
i) A gene of the genome as well as the associated protein are represented by a neuron of the equivalent neuronal network.
ii) The expression rate of a gene is represented by a non-negative activity of the equivalent neuron.
iii) The regulatory effect of the protein on a gene is represented by a synaptic connection from the neuron equivalent to the protein to the neuron equivalent to the gene.
iv) The type of regulatory effect (strengthening or inhibiting) is represented in the neuronal network by the leading sign and the strength of the associated synaptic weight.
v) In a further development it is also possible to represent a post-translational modification of a first protein by a second protein by a synaptic connection with multiplicative effect from the second neuron to the first neuron.
vi) In a further development an external influence on the regulatory network can be represented by an input node of the equivalent neuronal network.

The equivalent neuronal network is compared with the gene expression patterns determined and is adapted to these. From the adapted neuronal network the regulatory network of the cell investigated is deduced.

There is thus an equivalence relationship between the functional network of the genome and proteome on the one side and the neuronal network of the human brain on the other side, which both represent strongly networked closed-loop systems. This mapping brings about successful modeling of the functional network of proteins and genes.

The method allows the identification of target proteins on a systematic basis. The equivalence relationship described can be established between genetic and neuronal networks. The dynamic interaction of genes and regulatory proteins is thus modeled by a dynamic neuronal network. The method uses the information contained in the chronological sequence of the gene expression pattern for the identification of causal regulatory interrelationships.

As a rule genetic illnesses lead to complex malfunctions which however often only lead back to a few malfunctioning genes or proteins. Until now these key genes have not been known except in individual cases. Instead heuristic processes have been used in conventional target finding to search for targets for which regulation without medicament would restore the healthy gene expression pattern in the best possible way.

Recent estimates talk of 10,000 different proteins as possible targets in the human genome which it would not be practicable to thin out using a heuristic approach alone.

The model approach described here represents a powerful method for systematic target finding, that its for identification of one or more key genes or proteins which are located at the start of the regulation cascade and which for example introduce an organic development, regulate the regeneration capability of tissue but which are also responsible for mostly complex changes of gene expression patterns in the event of illness.

The method described allows a computer-based target finding which is able to analyze the large amount of data and the numerous and complex interrelationships.

It allows the following application areas to be specified:
Model-based support of research activities for decoding the human morphogenesis and there by the general principles of genetically controlled growth, regeneration and breakdown processes.
Support for the identification of target proteins which are fundamentally responsible for problems with growths e.g. unrestricted tumor growth and do not just represent one symptom. Novel methods for highly-sensitive early tumor diagnosis can be derived from this but also treatment methods such as selectively induced cell death (apoptosis) in tumor cells.
Support for the identification of regulatory proteins which intervene into growth and regeneration processes. This would overcome a significant hurdle on the way to induced regeneration of organs.

### BRIEF DESCRIPTION OF THE DRAWINGS

These and other objects and advantages of the present invention will become more apparent and more readily appreciated from the following description of the preferred embodiments, taken in conjunction with the accompanying drawings of which:

FIG. 1 a schematic diagram of the regulatory processes which determine the expression pattern of a cell;

FIG. 2 a schematic diagram of the networking of neurons;

FIG. 3 the potentials within a dendrite or a neuron as a function of the time and FIG. 4 a schematic diagram or a modulatory synapse.

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

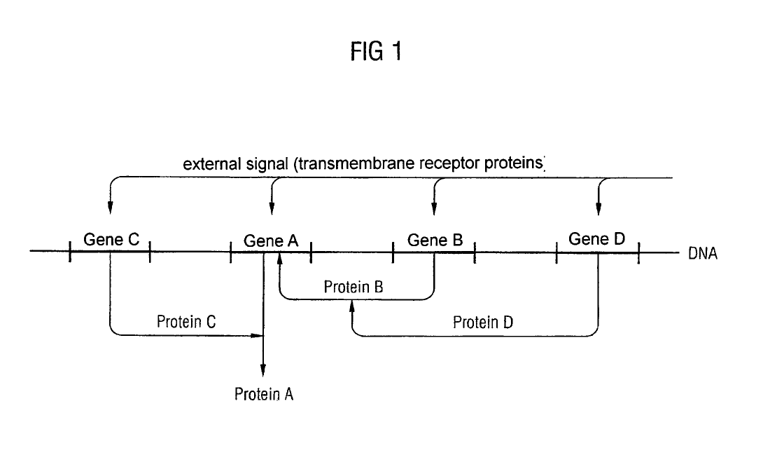

FIG. 1 illustrates the main interactions between genes and proteins of a section of DNA. The interactions are included as the basis for the description of the genomic regulatory network.

The top part of FIG. 1 shows schematically an external signal affecting the cell from the outside—within the framework of intercellular communication for example—which is accepted for example by a transmembrane receptor protein (e.g. from a calcium channel) and is transmitted in an appropriate manner into the inside of the cell, initiates the production of the genes A, B, C and D of the DNA section.

The option thus basically exists for influencing the expression rate of individual genes of a cell over the path mentioned from outside the cells.

A not necessarily contiguous section of the DNA is referred to as a gene which contains the genetic code for a protein or also for a group of proteins. In general the DNA features what are known as exons and introns. Exons represent parts of the DNA which actually encode a protein. Introns represent parts of the DNA which do not directly encode a protein. In a first approximation they have no function. Exons and introns alternate with each other in the DNA. If a gene is referred to as the quantity of the exons which together encode a specific protein, such a gene—as mentioned above—is as a rule not contiguous.

The production process of a protein from a gene, for example protein A, starting from gene A in FIG. 1, is referred to as the expression of this gene. The conversion of the DNA code of the gene into the chain of the amino acids of the protein is referred to as translation. The rate at which the protein A is produced in a given context is called its expression rate.

Not all genes are expressed in a cell. Instead different cell types are differentiated by their gene expression pattern. This then often also applies to the difference between diseased and healthy cells.

The expression pattern of a cell is determined by the regulatory processes shown schematically in FIG. 1. The regulatory processes are essentially determined by a few important interactions between proteins and genes as well as between the proteins themselves.

Thus the expression rate of a gene A can be regulated by the presence of another protein B, i.e. increased, reduced or brought to a standstill. In this example the protein B acts in a regulatory way on the gene A or the protein A. The protein components of activator complexes can for example be reckoned to be regulatory proteins. Regulatory proteins can operate on many target genes simultaneously.

A second type of interaction is a post-translational modification of proteins, i.e. the modification of proteins after their translation. As a rule the post-translational modification of a protein occurs directly after the translation, i.e. before the protein acts in the cell. Thus for example many proteins are phosphorylized or glycolyzed by specific enzymes, i.e. the target protein is put into its functional state by appending or splitting off chemical groups or is put into a state of in which it no longer has any effect. Post-translational modification can thus temporarily switch on or switch off the functions of a protein where necessary.

In FIG. 1 the protein A is what is referred to as an effector protein, i.e. it operates within the cell on other substances and not directly on the genome or the proteome. In FIG. 1 the protein C during the course of post-translational modification modifies the function of the effector protein A.

Protein B is a regulatory protein since it determines the expression rate of the protein A by interacting with that DNA section which contains the gene A. The protein D thus modifies the function of a regulatory protein (protein B) in the course of the post-translational modification.

Database

The nucleic acid sequence of the human DNA is very largely known. The genes encoded by the DNA have also been identified to an increasing extent. Not quite so complete is the knowledge about the proteins including the post translational modified proteins possibly produced by interactions between the proteins. In any event new sequencing and high-throughput screening processes allow further genes and proteins to be quickly identified.

A further important step for clarifying the expression pattern of a cell has been completed with the development of high-throughput hybridization techniques. With this method the expression rate of many 100 different genes is tested simultaneously on what is known as a microarray. With the aid of this method it is possible to determine the gene expression pattern of a cell.

To this end the mRNA (messenger RNAs) synthesized in the cell are determined as a rule. The mRNA is an intermediate product in the translation of the gene into a protein. The mRNA is thus a preliminary stage in the formation of the protein and points to the formation of the associated protein. The cell to be investigated is first isolated. Subsequently it is deduced. Suitable rationalization steps are used to isolate the mRNAs from the cell. Then the mRNA is translated using reverse transcriptasis into cDNA (complementary DNA). This is generally amplified by linear PCR (polymerase chain reaction). The cDNA thus obtained is analyzed with the aid of suitable microarrays, e.g. DNA chips, qualitatively or quantitatively. With modern microarrays the expression rates of 5,000 and more genes can be calibrated simultaneously.

Because of these improved techniques there is now comprehensive knowledge about the human genome and proteome as well as about the interactions between proteins and genes or between the proteins themselves.

Of particular importance are data records in which the chronological sequence of gene expression patterns in a tissue is stored. What are known as longitudinal hybridization studies, i.e. chronological sequences of gene expression patterns during the organ differentiation as part of the embryonal development is one example that might be mentioned. Time-resolved gene expression data also exists for the cell division cycle of single cell creatures and is also possible for more complex tissue.

Neuronal modeling of genome and proteome (cf. PCT03381)

A general outline of the modeling principle is given below. The basics are known from PCT03381.

The basic principle relates to establishing an equivalence relationship between the functional network of the genome and proteome on the one hand and the neuronal network of the human brain on the other hand, which both represent heavily networked closed-loop systems.

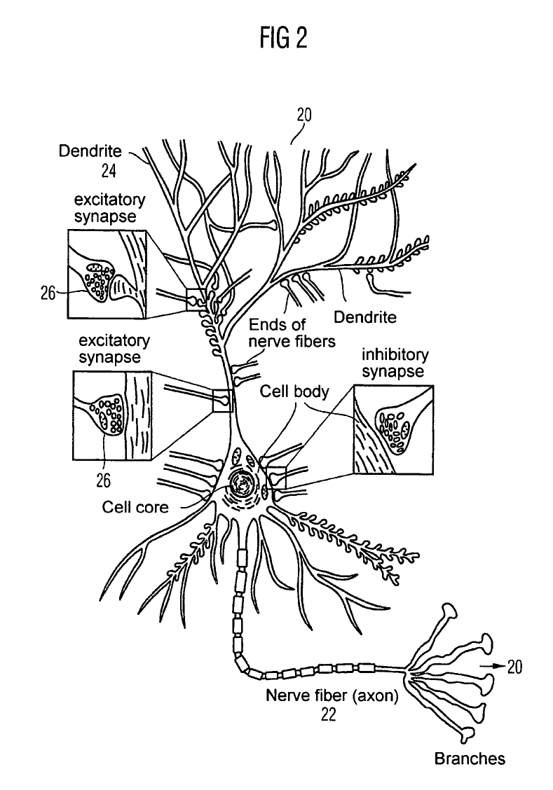

The neuronal network of the human brain is illustrated below in a plurality of fundamentals with reference to FIG. 2.

In the human brain there are around 100 billion nerve cells 20 (neurons), which each exchange information with tens of thousands of other nerve cells 20. The information passes from a neuron 20 via the axon 22 belonging to each neuron to another neuron 20. Each neuron has precisely one axon to send information to other neurons. In its further progress and axon typically branches around one thousand times, so that a neuron 20 can send information of via its axon 22 to around one thousand other neurons 20.

To receive information neurons 20 have dendrites 24. The axon 22 carrying information is connected via a synapse 26 with the dendrites 24. The information passes via this synapse 26 from the axon 22 into the dendrite 24 and thereby from the emitting neuron 22 to the downstream neuron. Between thousands and hundreds of thousands of axons 22 or synapses 26 can access a single dendrite 24 so that a downstream neuron 20 can receive signals from many 1000 upstream neurons.

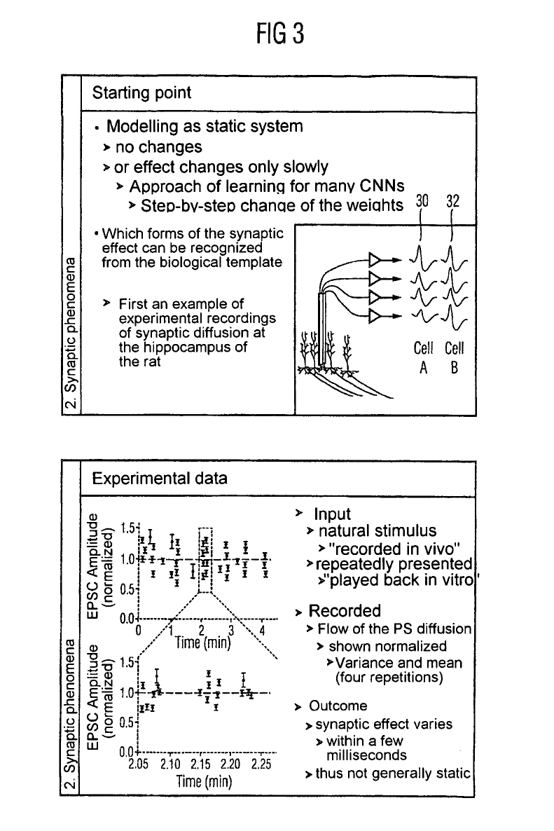

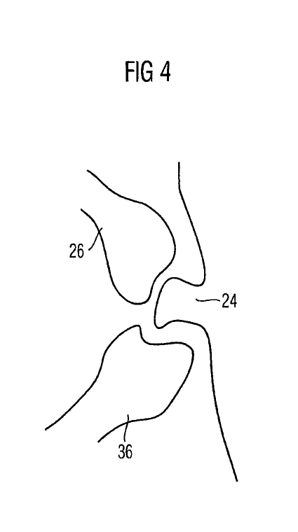

Reference is made to FIG. 3 below which shows the potentials within a dendrite or a neuron as a function of time. The information is exchanged between the neurons 20 in the form of action potentials (spikes) 30, which each neuron 20 emits via its axon 22. The spikes evoke renewed signals in the downstream neurons 20, the so-called post-synaptic potentials (PSPs) 32. The size of these PSPs depends on the transmission strength or the synaptic weight w of the synapse concerned.

The text below refers to FIG. 4. FIG. 4 shows dendrite 24, to which a first synapse 26 couples. A second synapse 36 is coupled to this first synapse 26. This second synapse 36 is called a modulatory synapse. If, with reference to FIG. 3 we designate the post-synaptic potential 32 as PSP which would form in dendrite 24 as a result of the effect of the first synapse 26 in the absence of the modulatory synapse 36, this can be represented by
PSP=W·ε(t),
with w, as defined above, representing the synaptic weight of the first synapse 26 and 8(t) the timing of the post-synaptic potential 32 in a suitable normalization.

If in addition the modulatory synapse 36 accesses the first synapse 26, this produces a modified post-synaptic potential PSP′ in the dendrite 24 which can be expressed by a multiplicative term act:
PSP′=act·w·ε(t)=act·PSP.

In this case act identifies the activity of the modulatory synapse 36.

For example dopaminergic synapses have a modulatory character in the central nervous system, that is in the brain and the spinal cord.

The neuronal activity of each neuron, i.e. the number of the spikes emitted for each unit of time, is produced—in simple terms—by a non-linear and chronologically non-local function of all incoming post-synaptic potentials. If this function exceeds a specific threshold value a spike 30 is initiated and transmitted via the axon 22.

Thus the biological neuronal network of the brain represents a complex non-linear system which also features a high networking density. To describe this system in a formal model neuro-information technology has developed powerful theories and algorithms in recent years (e.g. compartment model, spike response model, mean-field model, multi-modular neuro cognitive model, Bayes belief networks).

These theories or equations correspond in their structure to the equations derived above for reaction kinetics. Thus the regulatory network of genome and proteome of a cell can be mapped to an equivalent neuronal network as follows:
A gene A of the genome (understood here as that combination of exons which uniquely encode a protein) as well as the associated protein A are identified with a neuron A of the equivalent neuronal network. Since in the gene expression pattern only the mRNAs or cDNAs are qualitatively analyzed, it is also not possible at the level of the gene expression pattern to distinguish between genes and proteins just like that.
The expression rate of a gene A is expressed as a non-negative activity, e.g. the spike rate of the neuron A.
If a protein B acts in a regulatory manner on a downstream gene A, the equivalent neuronal network contains a synaptic connection from neuron B to the equivalent downstream neuron A.
The type of regulatory effect (strengthening or inhibiting) is specified in the neuronal network by the leading sign and the strength of the associated synaptic weight.
A post-translational modification of a protein by another protein, in FIG. 1 for example the modification of protein B by protein D, corresponds to the effect of a modulatory synapse in the central nervous system. Modulatory synapses are described in artificial neuronal networks by synaptic connections with multiplicative effect on other synapses. The equivalent reflection of a post-translational modification of the protein B by a protein D is thus a synaptic connection with multiplicative effect from neuron D to neuron B.
External signals are identified by input nodes of the equivalent neuronal network.

The equivalence relationship described can be established between genetic and neuronal networks. The dynamic interaction of genes and regulatory proteins is thus modeled by a dynamic neuronal network.

Networks of spiking neurons count as suitable neuronal algorithms but also mean-field models which take into account the explicit passage of time of the signal transmission between the neurons by the explicit description of the post-synaptic potentials. They allow the modeling of the development over time of the neuronal activities in the network as a result of external stimulation or intrinsic activity.

The development over time of the concentrations which is produced by the reaction kinetics between the molecules involved (e.g. between regulatory protein and DNA promoter) will thus be replaced by the time sequence of the activities of the neurons so that the resulting network model for simulating the timing development of gene expression patterns can be included.

The neuronal activities over time can be included for this type of neuronal network. Since the neuronal activity corresponds to the gene expression patterns, the two can be compared to each other. The neuronal network corresponds to a simulated gene expression pattern.

The object of the modeling is to determine the regulatory network underlying the expression sequence, i.e. to answer the following question: “Which neuronal networking structure with which weights and reaction constants is consistent with the observed gene expression sequence?”

To answer this question the network is trained with a method oriented to structured learning: An attempt is made to explain the observed behavior with as few regulatory connections as possible but also as well as possible, that is to find the simplest model consistent with the data.

A preferred optimization method minimizes the total deviation between measured and simulated gene expression patterns by using a “sparse prior”, that is an additional condition which penalizes the co-existence of many connections with small weights in favor of fewer regulatory connections. An option for implementing such a sparse prior is known to those skilled in the art.

Cross-validation and statistical optimization allow the uniqueness of the solution to be estimated as well as its ability to predict (generalization capability).

Causal relationships between genes but also the role of different genes can be taken from the trained network on the basis of the connection structure of the neurons. Thus an asymmetrical weight only from gene B to gene A indicates that gene B regulates gene A. At the same time in the model different genes or regulatory connections can be artificially switched off or switched on and the effects of the gene expression pattern with the target quantified, to identify the cause(s) of the illness-related changes of the gene expression (known as inverse modeling).

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

1-5. (canceled)

6. A method for identifying pharmaceutical targets, comprising:
determining a plurality of gene expression patterns for genes of similar cells, and for each gene expression pattern, determining an expression rate of the genes in a cell;
at least partially reconstructing a chronological sequence for the gene expression patterns;
forming a dynamic model of a regulatory network of genome and proteome for a cell using a neuronal network formed in the following manner:
representing a gene of the genome and its associated protein with a neuron in the neuronal network;
representing the expression rate of the gene with a non-negative activity of the neuron;
representing a regulatory effect of a first gene/protein on a second gene using a synaptic connection from a neuron representing the first gene/protein to a neuron representing the second gene; and
representing whether the regulatory effect is strengthening or inhibiting by changing the sign of the synaptic connection and by weighting the synaptic connection;
comparing the neuronal network with and adapting the neuronal network to each gene expression pattern; and
deducing the regulatory network based on the adapted neuronal network.

7. The method in accordance with claim 6, wherein
a post-translational modification of a first protein by a second protein is represented by a synaptic connection with a multiplicative effect from a second neuron to a first neuron.

8. The method in accordance with claim 6 wherein
an external influence on the regulatory network is represented by an input node in the neuronal network.

9. The method in accordance with claim 6 wherein
the neuronal network is adapted to each specific gene expression pattern so as to reduce a level of networking.

10. The method in accordance with claim 7 wherein
an external influence on the regulatory network is represented by an input node in the neuronal network.

11. The method in accordance with claim 10 wherein
the neuronal network is adapted to each specific gene expression pattern so as to reduce a level of networking.

12. The method in accordance with claim 7 wherein
the neuronal network is adapted to each specific gene expression pattern so as to reduce a level of networking.

13. The method in accordance with claim 8 wherein
the neuronal network is adapted to each specific gene expression pattern so as to reduce a level of networking.

14. A system for identifying pharmaceutical targets, comprising:
determining means for determining a plurality of gene expression patterns for genes of similar cells, with an expression rate of the genes in a cell being determined for each gene expression pattern, the determining means at least partially reconstructing a chronological sequence for the gene expression patterns in the cell;
modeling means for forming a dynamic model of a regulatory network of genome and proteome for the cell using a neuronal network formed in the following manner:
representing a gene of the genome and its associated protein with a neuron in the neuronal network;
representing the expression rate of the gene with a non-negative activity of the gene neuron;
representing a regulatory effect of a first gene/protein on a second gene using a synaptic connection from a neuron representing the first gene/protein to a neuron representing the second gene; and
representing whether the regulatory effect is strengthening or inhibiting by changing the sign of the synaptic connection and by weighting the synaptic connection; comparison means for comparing the neuronal network with each gene expression pattern;
adapting means for adapting the neuronal network to each gene expression pattern; and
deducing means for deducing the regulatory network based on the adapted neuronal network.
